(19) United States
(12) Patent Application Publication
(10) Pub. No.: US 2024/0180897 A1
(43) Pub. Date: Jun. 6, 2024

(54) METHODS AND COMPOSITIONS FOR TREATING PULMONARY HYPERTENSION

(71) Applicant: MannKind Corporation, Danbury, CT (US)

(72) Inventors: John J. Freeman, New Fairfield, CT (US); Marshall L. Grant, Newtown, CT (US); Jason J. Antunovich, Redding, CT (US); William Elliott Bay, Ridgefield, CT (US)

(21) Appl. No.: 18/437,733

(22) Filed: Feb. 9, 2024

Related U.S. Application Data
(62) Division of application No. 17/975,403, filed on Oct. 27, 2022.
(60) Provisional application No. 63/272,467, filed on Oct. 27, 2021.

(51) Int. Cl. A61K 31/495; A61K 9/00; A61K 31/191; A61P 9/12

(52) U.S. Cl. CPC A61K 31/495; A61K 9/0075; A61K 31/191; A61P 9/12

(57) ABSTRACT

Inhalable compositions for treating pulmonary hypertension comprising treprostinil, derivative thereof or analogs thereof and a method for treating pulmonary arterial hypertension and/or idiopathic pulmonary fibrosis are disclosed herein. Methods of manufacturing pharmaceutical compositions are also disclosed. Compositions are based on diketopiperazine powders for pulmonary inhalation.

### CROSS-REFERENCE TO RELATED APPLICATION

This application claims priority to U.S. Provisional Patent Application Ser. No. 63/272,467, filed Oct. 27, 2021, the contents of which is hereby incorporated by reference in its entirety.

### TECHNICAL FIELD

Inhalable compositions comprising treprostinil and a diketopiperazine, methods of manufacturing, and methods of using the compositions to treat lung disease are disclosed. In particular, the compositions are for treating pulmonary arterial hypertension and idiopathic pulmonary disease.

### BACKGROUND

Pulmonary arterial hypertension (PAH) is a complex, multifactorial, progressive syndrome characterized by persistent elevation of pulmonary arterial pressure and pulmonary vascular resistance (PVR) that leads to increase in right ventricular afterload and eventually culminates in right heart failure. Right ventricular failure limits cardiac output during exertion. The most common symptom at presentation is breathlessness, fatigue, angina, syncope, and abdominal distension, with impaired exercise capacity as a hallmark of the disease.

While there are currently various methods of treating PAH using tablets, nebulizers, injectables, and pumps to alleviate the symptoms of the disease, it would be advantageous to provide a patient suffering with PAH an easy and effective method to obtain their medications. Accordingly, new medications and methods for PAH treatment are needed to facilitate the administration of these products to a patient.

Drug delivery to lung tissue has been achieved using a variety of devices for inhalation, including, nebulizers and inhalers, such as metered dose inhalers and dry powder inhalers to treat local disease or disorders. Delivering powder formulation through inhalers can be accomplished with some ease depending on the type of compound to be formulated and the type of inhaler used. Some other compounds are more difficult to deliver due to certain properties, for example, some are hygroscopic, or hydrophobic, temperature sensitive or resistant, etc., and they are difficult to formulate with certain pharmaceutically acceptable carriers and/or excipients, due to stability, insolubility, viscosity, and other inherent chemical characteristics. Compounds that are difficult to formulate, which are difficult to solubilize include the prostacyclins and derivatives and salts thereof, including, treprostinil. Therefore, new processes for formulating medications for the treatment of disease, which are stable, can maintain therapeutically effective activity, preferably at room temperature, and can be stored for a long period of time.

### SUMMARY

Processes that facilitate the formulation of prostacyclin and analogs thereof, including, treprostinil and derivatives thereof, into dry powder compositions for inhalation and deep lung delivery are disclosed. A dry powder composition comprising, for example, treprostinil, or a derivative thereof, or combinations thereof, can be used for the treatment of disease, including, pulmonary arterial hypertension and/or idiopathic pulmonary fibrosis is disclosed.

In one embodiment, the process for making a dry powder composition comprising a hydrophobic compound comprises preparing a suspension of a dry powder composition comprising crystalline particles of a diketopiperazine, including (E)-3,6-bis[4-(N-carbonyl-2-propenyl)amidobutyl]-2,5-diketopiperazine, and an acetic acid solution in a high shear mixer at a temperature ranging from 13° C. to about 20° C. while mixing the suspension; washing the suspension in water; pelletizing the suspension in a cryogranulator, and drying the suspension in a lyophilizer to collect the crystalline particles of the diketopiperazine. In this embodiment, the process comprises resuspending a dry powder comprising dried crystalline particles of a diketopiperazine in a solution of deionized water and an alcohol to contain from about 0.5% to about 2% solid in the suspension, or from about 1% to about 4% solid in the suspension, or from about 1% to about 10% solid in the suspension or higher than 10%; preparing a solution comprising treprostinil in a diluted ethanol in water solution, or absolute ethanol, and mixing the solution comprising the treprostinil to the crystalline particles of diketopiperazine in suspension; and spray-drying the suspension to form a dry powder composition. In one embodiment, the process comprises adding more ethanol to the suspension to attain a density of the ethanol/water that is closer to the density of treprostinil, which causes the treprostinil to be less buoyant and more uniformly dispersed in the suspension.

Methods for using the compositions in the treatment of pulmonary hypertension are also disclosed. In embodiments herewith, a method comprises providing a treprostinil composition in a dry powder inhaler comprising a replaceable single dose cartridge comprising a dry powder for inhalation for delivery to the lungs for local, or systemic delivery into the pulmonary circulation. The dry powder inhaler is a breath-powered inhaler which is compact, reusable or disposable, has various shapes and sizes, and comprises a system of airflow conduit pathways for the effective and rapid delivery of powder medicament to the lungs and the systemic circulation. In one embodiment, a dry powder composition for inhalation comprising treprostinil or a salt thereof, is provided into the respiratory system in less than 10 seconds, or less than 5 seconds, or less than 3 seconds and the treprostinil is detected at peak concentrations in the blood of the subject treated in less than 30 minutes, having a median Tmax (treprostinil max) of about 10 minutes or less.

In a particular embodiment, the method of treating pulmonary arterial hypertension utilizes a drug delivery system, which is designed for drug delivery to the lungs, including by inhalation, for rapid delivery and onset of action of the active agent being delivered to target tissues using the arterial circulation in the lungs. In this method, the active agent can reach its target site in a therapeutically effective manner. In one embodiment, the method of treatment of PAH and/or idiopathic pulmonary disease is with the present composition using an oral inhalation device that can deliver the active agent deep into the lung's pulmonary alveoli.

In one embodiment, the method comprises administering a stable pharmaceutical composition comprising, one or more active agents, including, a vasodilator, including, sildenafil, tadalafil, vardenafil, a prostaglandin or an analog thereof, for example, treprostinil or a pharmaceutically acceptable salt thereof, including treprostinil sodium, for treating PAH and delivering the treprostinil into the systemic circulation of the lungs of a subject by pulmonary inhalation using a dry powder inhaler. In one embodiment, the method comprises providing to a patient in need of treatment a dry powder inhaler comprising treprostinil in a stable dry powder formulation, and administering the active agent by oral inhalation.

In one embodiment, the drug delivery system comprises a dry powder inhaler comprising a diketopiperazine-based drug formulation for delivering small molecules, for example, a prostaglandin, or analogs thereof including, treprostinil and/or protein-based products for treating PAH. The method provides advantages over typical methods of drug delivery, such as, oral tablet and subcutaneous and intravenous injectable/infusion drug products that are sensitive to degradation and/or enzymatic deactivation.

In certain embodiments disclosed herein, a method for providing a prostaglandin formulation to a patient in need thereof is disclosed, the method comprising, selecting a patient to be treated for PAH, and administering to the patient a dry powder formulation comprising treprostinil, wherein the treprostinil is combined with a diketopiperazine, including, (E)-3,6-bis[4-(N-carbonyl-2-propenyl)amidobutyl]-2,5-diketopiperazine to produce a pharmaceutical formulation or composition suitable for pulmonary inhalation, and delivering the treprostinil formulation to the patient's lungs the composition using a breath-powered dry powder inhaler in one or more than one breaths per treatment session. In this and other embodiments, the dry powder formulations can be provided more than once a day as needed by the patient and the composition is provided in a reconfigurable cartridge comprising from about 1 μg to about 200 μg of treprostinil in the dry powder formulation per dose, from 10 μg to about 100 μg, from about 100 μg to about 150 μg, from about 150 μg to about 300 μg of Treprostinil, a derivative thereof, an analog thereof or combinations thereof.

In certain embodiments, the dry powder formulation can comprise from about 10 μg to about 300 μg of treprostinil per treatment dose in a cartridge or capsule. In one embodiment, a cartridge for single use can comprise from about 10 μg to about 90 μg of treprostinil for at least one inhalation. In some embodiments, the dry powder formulation is delivered using at least one inhalation per use or dose. In this and other embodiments, the dry powder formulation is delivered to a patient in less than 10 seconds, or less than 8 seconds, or less than 6 seconds per inhalation or breath. In one embodiment, the pharmaceutical dry powder composition comprises microcrystalline particles of fumaryl diketopiperazine wherein the particles have a specific surface area ranging from about 59 m²/g to about 63 m²/g and have a pore size ranging from about 23 nm to about 30 nm.

Also disclosed herein is a method for treating pulmonary arterial hypertension disease or disorder comprising selecting a patient to be treated with pulmonary arterial hypertension of functional classification I, II or III, or a patient with PAH which exhibits a condition treatable with an active agent, including treprostinil, epoprostenol, bosentan, ambrisentan, macisentan, sildenafil, tadalafil, riociguat and the like, or combinations thereof, which patients are typically treated only by oral or injectable administration; replacing the aforementioned therapy with an inhalation therapy comprising providing the patient with an inhaler comprising the active agent in a stable dry powder composition for treating the disease or disorder; wherein the stable dry powder composition comprises the active agent and a diketopiperazine; and administering the stable dry powder composition to the patient by pulmonary inhalation; thereby treating the disease or condition.

In an exemplary embodiment, the formulation for treating pulmonary arterial hypertension comprises treprostinil in an amount up to 200 μg per dose, for example, amounts of 1 μg, 5 μg, 10 μg, 15 μg, 20 μg, 30 μg, 60 μg, 90 μg, 100 μg, 120 μg, 150 μg, 180 μg, or 200 μg, and one or more pharmaceutically acceptable carriers and/or excipients per dose are to be administered to a subject. In this embodiment, the pharmaceutically acceptable carrier and/or excipient can be formulated for oral inhalation and can form particles, for example, a diketopiperazine, including, fumaryl diketopiperazine, sugars such as mannitol, xylitol, sorbitol, and trehalose; amino acids, including, glycine, leucine, isoleucine, methionine; surfactants, including, polysorbate 80; cationic salts, including, monovalent, divalent and trivalent salts, including, sodium chloride, potassium chloride, magnesium chloride, and zinc chloride; buffers such as citrates and tartrates, or combination of one or more carriers and/or excipients and the like. In a particular embodiment, the formulation comprises a dry powder comprising treprostinil, a sugar and an amino acid, wherein the sugar is mannitol or trehalose; and the amino acid is leucine or isoleucine and a cationic salt. In certain embodiments, the formulation can further comprise sodium chloride, potassium chloride, magnesium chloride or zinc chloride, sodium citrate, sodium tartrate, or combinations thereof.

In an exemplary embodiment, the treprostinil dose is administered using a dry powder inhaler for oral inhalation using one or more inhalations from a dry powder inhaler. In this embodiment, a treprostinil inhalation powder dose is provided to a patient suffering with pulmonary arterial hypertension and in need of treatment; wherein the dry powder inhaler comprises a container including, a cartridge, and the container or cartridge comprises the dry powder comprising treprostinil and (E)-3,6-bis[4-(N-carbonyl-2-propenyl)amidobutyl]-2,5-diketopiperazine to be administered in multiple daily doses for a period of six months and the treprostinil is administered by oral inhalation at an earlier time in the course of the disease to patients with Functional Class I, II or III as a first line monotherapy. In this and other embodiments, the treprostinil composition comprises single dose capsules or cartridges comprising 8 μg, 16 μg, 32 μg, 64 μg, 80 μg, or a combination of cartridges thereof comprising the treprostinil or a salt thereof which can be provided to the patient in a blister package for ease of use. In certain embodiments, the method for treating disease, including but not limited to, PAH, interstitial lung disease (ILD), and/or pulmonary fibrosis, comprises administering to a patient in need of treatment, one or more capsules or cartridges to attain the required dosing for an individual patient. In certain embodiments, the method comprises administering a predetermined dose of an inhalable dry powder comprising treprostinil to a patient using a dry powder inhaler once or more than once per day as determined by the physician. In particular, the treatment regime can be administered twice a day. In some embodiments, the method comprises administering a predetermined dose of an inhalable dry powder per cartridge using a single inhalation per cartridge dose.

In one embodiment, a method for treating pulmonary arterial hypertension is provided comprising providing a patient in need of treatment a monotherapy using an inhalable dry powder comprising treprostinil and a pharmaceutically acceptable carrier, and/or excipient by oral inhalation using a dry powder inhaler and a container comprising the inhalable dry powder and administering the dry powder formulation to the patient. In some embodiments, the treprostinil formulation comprises fumaryl diketopiperazine particles.

In one embodiment, a method for treating pulmonary arterial hypertension is provided comprising providing a patient in need of treatment a combination therapy using an inhalable dry powder comprising treprostinil and fumaryl diketopiperazine, and administering separately in combination with orally administered drugs selected from prostacyclin analogues, endothelin receptor antagonists (ERAs), including bosentran, ambrisentran and macitentan, soluble guanine cyclase agonists/stimulators such as riociguat, and PDE-5 inhibitors, including sildenafil, vardenafil and tadalafil.

In another embodiment, a dry powder comprising treprostinil and fumaryl diketopiperazine can also be administered as a part of up-front combination therapy with an oral agent. In an alternate embodiment, an inhalable treprostinil composition comprising a dose of fumaryl diketopiperazine and treprostinil powder, wherein treprostinil is in an amount from about 1 μg to about 200 μg can be administered in combination with an oral agent such as a PDE-5 inhibitor, or an endothelin receptor antagonist and/or the combination therapy can also be administered to replace continuously parenteral infusion of prostacyclin analogs in patients with severe disease and classified in WHO Functional class IV. Phosphodiesterase inhibitors, including PDE-5 inhibitors can also be formulated for inhalation alone, or in combination with the treprostinil and can be administered subsequently if administered alone, as a combination therapy.

In another embodiment, the inhalation system comprises a breath-powered dry powder inhaler, a container or cartridge containing a dry powder, for delivering an active agent to the pulmonary tract and lungs, including a medicament, wherein the medicament can comprise, for example, an inhalable drug formulation for pulmonary delivery such as a composition comprising a diketopiperazine in a crystalline powder form that self-assembles in a suspension, an amorphous powder form, and/or a microcrystalline powder form comprising crystallites that do not self-assemble in suspension, or combinations thereof, and an active agent, including, treprostinil, sildenafil, vardenafil, tadalafil, or combinations thereof. Cartridges or capsules can be assembled into blister packages for easy access to the treatment.

In alternate embodiments, the dry powder for inhalation may be formulated with other carriers and/or excipients other than diketopiperazines, for example a sugar, including trehalose; buffers, including sodium citrate; salts, including, sodium chloride and zinc chloride, and one or more active agents, including, treprostinil, vardenafil, and sildenafil.

In embodiments herewith, the method of treating PAH comprises, administering to a patient with moderate to severe PAH a dry powder formulation comprising treprostinil and a pharmaceutically acceptable carrier and/or excipient in an amount up to 200 μg of treprostinil using a dry powder inhaler comprising a movable member for loading a container comprising the pharmaceutical composition and the movable member can configure a container to attain a dosing configuration from a container loading configuration so that inhaler creates an airflow through the inhaler during an inhalation maneuver to allow the contents of the container to enter the airflow path and greater than 60% of a dry powder dose in the container is delivered to the lungs in a single inhalation.

In some embodiments, the treatment regimen with an inhalation dry powder depends on the patient's need and can be one inhalation to replace each of a nebulization session performed with standard therapy, including, at least one to four inhalations per day depending on the severity of disease.

In one embodiment, a kit for providing the present compositions to a patient being treated for PAH/ILD disease is also disclosed, which comprises, inhalers, blisters containing various doses of the treprostinil compositions and instructions for use. In one embodiment, a kit can comprise a single blister type comprising a dose or combinations of blisters with various dose contents depending on the patient's need and treatment requirements.

### BRIEF DESCRIPTION OF THE DRAWINGS

FIG. 1 depicts a graph of data comparison on the relationship between treprostinil AUC0-5 (area under the curve) and increasing dose administered to patients in the study receiving treprostinil inhalation powder (TIP=TreT) administered by a dry powder inhaler and by standard therapy with TYVASO® administered by nebulization. As can be seen in the graph, both data sets indicate that administration of the dose with either method is linear with increasing amounts of treprostinil.

FIG. 2 depicts a graph of data comparison on the relationship between treprostinil Cmax (maximal concentration in blood) and increasing dose administered to patients in the study receiving treprostinil inhalation powder (TIP=TreT) administered by a dry powder inhaler and by standard therapy with TYVASO® administered by nebulization

FIG. 3 depicts a graph of the change from Baseline in 6MWD test for subjects treated with the present treprostinil inhalation powder (TIP), which demonstrates overall a significant improvement (8 meter increase; p=0.0217) at Week 3 of treatment and throughout the 59 weeks of the study.

### DETAILED DESCRIPTION

In embodiments disclosed herein, dry powder compositions and dry powder inhalers comprising a container or a cartridge for delivering dry powders including pharmaceutical medicaments to a subject by oral inhalation are described. In one embodiment, the dry powder inhaler is a breath-powered, dry powder inhaler, and the container or cartridge is designed to contain an inhalable dry powder, including, but not limited, to pharmaceutical formulations comprising an active ingredient, including a pharmaceutically active substance, and optionally, a pharmaceutically acceptable carrier. In particular, the dry powder inhalers are for the treatment of pulmonary arterial hypertension.

The dry powder inhalers are provided in various embodiments of shapes and sizes, and can be reusable, easy to use, inexpensive to manufacture and/or produced in high volumes in simple steps using plastics or other acceptable materials. Various embodiments of the dry powder inhalers are provided herein and in general, the inhalation systems comprise inhalers, powder-filled cartridges, and empty cartridges. The present inhalation systems can be designed to be used with any type of dry powder. In one embodiment, the dry powder is a relatively cohesive powder which requires optimal deagglomeration conditions. In one embodiment, the inhalation system provides a re-useable, miniature breath-powered inhaler in combination with single-use cartridges containing pre-metered doses of a dry powder formulation. The inhaler can deliver a dry powder dose in a single inhalation to a patient in treating pulmonary arterial hypertension in less than 10 seconds. In particular embodiments, oral inhalation can deliver greater than 60% of a powder dose in less than 6 seconds, in less than 4 seconds and in less than 2 seconds.

As used herein the term “a unit dose inhaler” refers to an inhaler that is adapted to receive a single enclosure, cartridge or container comprising a dry powder formulation and delivers a single dose of a dry powder formulation by inhalation from a single container to a user. It should be understood that in some instances multiple unit doses will be required to provide a user with a specified dosage.

As used herein a “cartridge” is an enclosure configured to hold or contain a dry powder formulation, a powder containing enclosure, which has a cup or container and a lid. The cartridge is made of rigid materials, and the cup or container is moveable relative to the lid in a translational motion or vice versa.

As used herein a “powder mass” is referred to an agglomeration of powder particles or agglomerate having irregular geometries such as width, diameter, and length.

As used herein a “unit dose” refers to a pre-metered dry powder formulation for inhalation. Alternatively, a unit dose can be a single enclosure including a container having a single dose or multiple doses of formulation that can be delivered by inhalation as metered single amounts. A unit dose enclosure/cartridge/container contains a single dose. Alternatively, it can comprise multiple individually accessible compartments, each containing a unit dose.

As used herein, the term “about” is used to indicate that a value includes the standard deviation of error for the device or method being employed to determine the value.

As used herein, the term “microparticle” refers to a particle with a diameter of about 0.5 to about 1000 μm, irrespective of the precise exterior or interior structure. Microparticles having a diameter of between about 0.5 and about 10 microns can reach the lungs, successfully passing most of the natural barriers. A diameter of less than about 10 microns is required to navigate the turn of the throat and a diameter of about 0.5 μm or greater is required to avoid being exhaled. To reach the deep lung (or alveolar region) where most efficient absorption is believed to occur, it is preferred to maximize the proportion of particles contained in the “respirable fraction” (RF), generally accepted to be those particles with an aerodynamic diameter of about 0.5 to about 6 μm, though some references use somewhat different ranges, as measured using standard techniques, for example, with an Anderson Cascade Impactor. Other impactors can be used to measure aerodynamic particle size such as the NEXT GENERATION IMPACTOR™ (NGI™, MSP Corporation), for which the respirable fraction is defined by similar aerodynamic size, for example <6.4 μm. In some embodiments, a laser diffraction apparatus is used to determine particle size, for example, the laser diffraction apparatus disclosed in U.S. Pat. No. 8,508,732, which disclosure is incorporated herein in its entirety for its relevant teachings related to laser diffraction, wherein the volumetric median geometric diameter (VMGD) of the particles is measured to assess performance of the inhalation system. For example, in various embodiments cartridge emptying of ≥80%, 85%, or 90% and a VMGD of the emitted particles of <12.5 μm, <7.0 μm, or <4.8 μm can indicate progressively better aerodynamic performance.

Respirable fraction on fill (RF/fill) represents the percentage (%) of powder in a dose that is emitted from an inhaler upon discharge of the powder content filled for use as the dose, and that is suitable for respiration, i.e., the percent of particles from the filled dose that are emitted with sizes suitable for pulmonary delivery, which is a measure of microparticle aerodynamic performance. As described herein, a RF/fill value of 40% or greater than 40% reflects acceptable aerodynamic performance characteristics. In certain embodiments disclosed herein, the respirable fraction on fill can be greater than 50%. In an exemplary embodiment, a respirable fraction on fill can be up to about 80%, wherein about 80% of the fill is emitted with particle sizes <5.8 μm as measured using standard techniques.

As used herein, the term “dry powder” refers to a fine particulate composition that is not suspended or dissolved in a propellant, or other liquid. It is not meant to necessarily imply a complete absence of all water molecules.

As used herein, “amorphous powder” refers to dry powders lacking a definite repeating form, shape, or structure, including all non-crystalline powders.

The present disclosure also provides improved powders comprising microcrystalline particles, compositions, methods of making the particles, and therapeutic methods that allow for improved delivery of drugs to the lungs for treating diseases and disorders in a subject. Embodiments disclosed herein achieve improved delivery by providing crystalline diketopiperazine compositions comprising microcrystalline diketopiperazine particles having high capacity for drug adsorption yielding powders having high drug content of one or more active agents. Powders made with the present microcrystalline particles can deliver increased drug content in lesser amounts of powder dose, which can facilitate drug delivery to a patient. The powders can be made by various methods including, methods utilizing surfactant-free solutions or solutions comprising surfactants depending on the starting materials.

In alternate embodiments disclosed herein, the drug delivery system can comprise a dry powder for inhalation comprising a plurality of substantially uniform, microcrystalline particles, wherein the microcrystalline particles can have a substantially hollow spherical structure and comprise a shell which can be porous comprising crystallites of a diketopiperazine that do not self-assemble in a suspension or in solution. In certain embodiments, the microcrystalline particles can be substantially hollow spherical and substantially solid particles comprising crystallites of the diketopiperazine depending on the drug and/or drug content provided and other factors in the process of making the powders. In one embodiment, the microcrystalline particles comprise particles that are relatively porous, having average pore volumes of about 0.43 cm³/g, ranging from about 0.4 cm³/g to about 0.45 cm³/g, and average pore size ranging from about 23 nm to about 30 nm, or from about 23.8 nm to 26.2 nm as determined by BJH adsorption.

Certain embodiments disclosed herein comprise dry powders comprising a plurality of substantially uniform, microcrystalline particles, wherein the particles have a substantially spherical structure comprising a shell which can be porous, and the particles comprise crystallites of a diketopiperazine that do not self-assemble in suspension or solution and have a volumetric median geometric diameter less than 5 μm; or less than 2.5 μm and comprise an active agent.

In a particular embodiment herein, up to about 92% of the microcrystalline particles have a volumetric median geometric diameter of 5.8 μm. In one embodiment, the particle's shell is constructed from interlocking diketopiperazine microcrystals having one or more drugs adsorbed on their surfaces. In some embodiments, the particles can entrap the drug in their interior void volume and/or combinations of the drug adsorbed to the crystallites' surface and drug entrapped in the interior void volume of the spheres.

In certain embodiments, a diketopiperazine composition comprising a plurality of substantially uniformly formed, microcrystalline particles is provided, wherein the particles have a substantially hollow spherical structure and comprise a shell comprising crystallites of a diketopiperazine that do not self-assemble; wherein the particles are formed by a method comprising the step of combining diketopiperazine having a trans isomer content ranging from about 45% to 65% in a solution and a solution of acetic acid without the presence of a surfactant and concurrently homogenizing in a high shear mixer at high pressures of up to 2,000 psi to form a precipitate; washing the precipitate in suspension with deionized water; concentrating the suspension and drying the suspension in a spray drying apparatus. The microcrystalline particles can be pre-formed without for later used, or combined with an active agent in suspension prior to spray drying.

The method can further comprise the steps of adding with mixing a solution comprising an active agent or an active ingredient such as a drug or bioactive agent along with other pharmaceutically acceptable carriers and/or excipients prior to the spray drying step so that the active agent or active ingredient is adsorbed and/or entrapped on or within the particles. Particles made by this process can be in the submicron size range prior to spray-drying.

In certain embodiments, a diketopiperazine composition comprising a plurality of substantially uniformly formed, microcrystalline particles is provided, wherein the particles have a substantially hollow spherical structure and comprise a shell comprising crystallites of a diketopiperazine that do not self-assemble, and the particles have a volumetric mean geometric diameter less than equal to 5 μm; wherein the particles are formed by a method comprising the step of combining diketopiperazine in a solution and a solution of acetic acid without the presence of a surfactant and concurrently homogenizing in a high shear mixer at high pressures of up to 2,000 psi to form a precipitate; washing the precipitate in suspension with deionized water; concentrating the suspension and drying the suspension in a spray drying apparatus.

The method can further comprise the steps of adding with mixing a solution comprising an active agent or an active ingredient such as a drug or bioactive agent prior to the spray drying step so that the active agent or active ingredient is adsorbed and/or entrapped on or within the particles. Particles made by this process can be in the submicron size range prior to spray-drying.

In certain embodiments, a diketopiperazine composition comprising a plurality of substantially uniformly formed, microcrystalline particles is provided, wherein the microcrystalline particles have a substantially hollow spherical structure and comprise a shell comprising crystallites of a diketopiperazine that do not self-assemble, and the particles have a volumetric mean geometric diameter less than equal to 5 μm; wherein the particles are formed by a method comprising the step of combining diketopiperazine in a solution and a solution of acetic acid without the presence of a surfactant and without the presence of an active agent, and concurrently homogenizing in a high shear mixer at high pressures of up to 2,000 psi to form a precipitate; washing the precipitate in suspension with deionized water; concentrating the suspension and drying the suspension in a spray drying apparatus.

In certain embodiments wherein the starting material comprising the active ingredient is an extract exhibiting a high degree of viscosity, or a substance having a honey like viscous appearance, the microcrystalline particles are formed as above and by washing them in water using tangential flow filtration prior to combining with the extract or viscous material. After washing in water, the resultant particle suspension is lyophilized to remove the water and re-suspended in an alcohol solution, including ethanol or methanol prior to adding the active ingredient as a solid, or in a suspension, or in solution. In one embodiment, optionally, the method of making the composition comprises the step of adding any additional excipient, including one or more, amino acid, such as leucine, isoleucine, norleucine, methionine or one or more phospholipids, for example, 1,2-dipalmitoyl-sn-glycero-3-phosphocholine (DPPC) or 1,2-distearoyl-sn-glycero-3-phosphocholine (DSPC), concurrently with the active ingredient or subsequent to adding the active ingredient, and prior to spray drying. In certain embodiments, forming the composition comprises the step wherein the extract comprising desired active agents is optionally filtered or winterized to separate and remove layers of unwanted materials such as lipids to increase its solubility.

The method can further comprise the steps of adding a solution with mixing to the mixture, and wherein the mixing can optionally be performed with or without homogenization in a high shear mixer, wherein the solution comprises an active agent or an active ingredient such as a drug or bioactive agent prior to the spray drying step so that the active agent or active ingredient is adsorbed and/or entrapped within or on the surface of the particles. Particles made by this process can be in the submicron size range prior to spray-drying, or the particles can be formed from the solution during spray-drying.

In some embodiments herewith, the drug content can be delivered on crystalline powders using FDKP and which are lyophilized or sprayed dried at contents to about 10%, or about 20%, or about 30% or higher. In embodiments using microcrystalline particles formed from FDKP, or FDKP disodium salt, and wherein the particles do not self-assemble and comprise submicron size particles, drug content can typically be greater than 0.01% (w/w). In one embodiment, the drug content to be delivered with the microcrystalline particles of from about 0.01% (w/w) to about 75% (w/w); from about 1% to about 50% (w/w), from about 10% (w/w) to about 25% (w/w), or from about 10% to about 20% (w/w), or from 5% to about 30%, or greater than 25% depending on the drug to be delivered. An example embodiment wherein the drug is a peptide such as insulin, the present microparticles typically comprise approximately 10% to 45% (w/w), or from about 10% to about 20% (w/w) insulin. In certain embodiments, the drug content of the particles can vary depending on the form and size of the drug to be delivered.

In an exemplary embodiment, the composition comprises a dry powder comprising microcrystalline particles of (E)-3,6-bis[4-(N-carbonyl-2-propenyl)amidobutyl]-2,5-diketopiperazine, wherein the treprostinil is adsorbed to the particles and wherein the content of the treprostinil in the composition comprises up to about 20% (w/w) and ranges from about 0.5% to about 10% (w/w), or from about 1% to about 5% (w/w) of the dry powder. In one embodiment, the composition herein can comprise other excipients suitable for inhalation such as amino acids including methionine, isoleucine and leucine. In this embodiment, the treprostinil composition can be used in the prevention and treatment of pulmonary hypertension by self-administering an effective dose comprising about 1 mg to 15 mg of a dry powder composition comprising microcrystalline particles of (E)-3,6-bis[4-(N-carbonyl-2-propenyl)amidobutyl]-2,5-diketopiperazine and treprostinil in a single inhalation. In a particular embodiment, the treprostinil content in the formulation can be from about 1 μg to about 200 μg. In one embodiment, the dry powder content of the cartridges comprising treprostinil can be 20 μg, 30 μg, 60 μg, 90 μg, 120 μg, 150 μg, 180 μg, or 200 μg. In other embodiments, the dry powder content of the cartridges comprising treprostinil can be about 20 μg, about 30 μg, about 60 μg, about 90 μg, about 120 μg, about 150 μg, about 180 μg, or about 200 μg, between about 20 μg to about 60 μg, between about 50 μg to about 90 μg, between about 60 μg to about 120 μg, between about 90 μg to about 120 μg, between about, 100 μg to about 150 μg, between about 120 μg to about 150 μg, between about 120 μg to about 180 μg, between about 150 μg to 180 μg, or between about 180 μg to 200 μg.

In alternate embodiments, the pharmaceutically acceptable carrier for making dry powders can comprise any carriers or excipients useful for making dry powders and which are suitable for pulmonary delivery. Example of pharmaceutically suitable carriers and excipients include, sugars, including saccharides and polysaccharides, such as lactose, mannose, sucrose, mannitol, trehalose; citrates, amino acids such as glycine, L-leucine, isoleucine, trileucine, tartrates, methionine, vitamin A, vitamin E, zinc citrate, sodium citrate, trisodium citrate, sodium tartrate, sodium chloride, zinc chloride, zinc tartrate, polyvinylpyrrolidone, polysorbate 80, phospholipids including diphosphotidylcholine and the like.

In one embodiment, a method of self-administering a dry powder formulation to one's lung(s) with a dry powder inhalation system is also provided. The method comprises: obtaining a dry powder inhaler in a closed position and having a mouthpiece; obtaining a cartridge comprising a pre-metered dose of a dry powder formulation in a containment configuration; opening the dry powder inhaler to install the cartridge; closing the inhaler to effectuate movement of the cartridge to a dosing position; placing the mouthpiece in one's mouth and, inhaling once deeply to deliver the dry powder formulation.

In still yet a further embodiment, a method of treating obesity, hyperglycemia, insulin resistance, pulmonary hypertension, anaphylaxis, and/or diabetes is disclosed. The method comprises the administration of an inhalable dry powder composition or formulation comprising, for example, a diketopiperazine having the formula (E)-3,6-bis[4-(N-carbonyl-2-propenyl)amidobutyl]-2,5-diketopiperazine. In this embodiment, the dry powder composition can comprise a diketopiperazine salt. In still yet another embodiment, there is provided a dry powder composition or formulation, wherein the diketopiperazine is (E)-3,6-bis[4-(N-carbonyl-2-propenyl)amidobutyl]-2,5-diketopiperazine, with or without a pharmaceutically acceptable carrier, or excipient.

An inhalation system for delivering a dry powder formulation to a patient's lung(s) is provided, the system comprising a dry powder inhaler configured to have flow conduits with a total resistance to flow in a dosing configuration ranging in value from 0.065 to about 0.200 (√kPa)/liter per minute. The dry powder inhaler can be provided comprising a dry powder formulation for single use that can be discarded after use, or with individual doses that are replaceable in a multiple use inhaler and the individual dose enclosures or containers can be discarded after use.

In one embodiment, a dry powder inhalation kit is provided comprising a dry powder inhaler as described above, one or more medicament cartridges comprising a dry powder formulation for treating a disorder or disease such as respiratory tract and lung disease, including pulmonary arterial hypertension, cystic fibrosis, respiratory infections, cancer, and other systemic diseases, including, endocrine disease, including, diabetes and obesity.

Methods of treating a disease or disorder in a patient with the dry powder inhaler embodiments disclosed herewith is also provided. The method of treatment comprises providing to a patient in need of treatment a dry powder inhaler comprising a cartridge containing a dose of an inhalable formulation comprising an active ingredient selected from the group as described above and a pharmaceutical acceptable carrier and/or excipient; and having the patient inhale through the dry powder inhaler deeply for about 3 to 4 seconds to deliver the dose. In this method, the patient can resume a normal breathing pattern thereafter. In all embodiments disclosed herein, the patient can resume a normal breathing pattern thereafter.

A process for making a pharmaceutical composition comprising treprostinil is disclosed. The process comprises making a dry powder composition for oral inhalation comprising treprostinil in an amount from 0.25% (w/w) to about 10% (w/w), or from about 1% (w/w) to about 5% (w/w) in the composition and microcrystalline particles of (E)-3,6-bis[4-(N-carbonyl-2-propenyl)amidobutyl]-2,5-diketopiperazine. In one embodiment, the process can be discontinuous comprising preparing a suspension of a dry powder composition comprising crystalline particles of a diketopiperazine, including (E)-3,6-bis[4-(N-carbonyl-2-propenyl)amidobutyl]-2,5-diketopiperazine, and an acetic acid solution in a high shear mixer at a temperature ranging from 13° C. to about 20° C. while mixing the suspension; washing the suspension in deionized water to remove the acetic acid; pelletizing the suspension in a cryogranulator, and drying the suspension in a lyophilizer to collect the crystalline particles of the diketopiperazine. In this embodiment, the dry powder pellets can be stored at room temperature or refrigerated until used. In another embodiment, the process can be continuous.

In one embodiment, the process comprises, resuspending a dry powder comprising dried microcrystalline particles of a diketopiperazine in a solution of deionized water and an alcohol to contain from about 0.5% to about 2% solid in the suspension, or from about 1% to about 4% solid in the suspension, or from about 1% to about 10% solid in the suspension or higher than 10%; preparing a solution comprising treprostinil in diluted ethanol in a water solution, or absolute ethanol, and mixing the solution comprising the treprostinil to the crystalline particles of diketopiperazine in suspension, and spray-drying the suspension to form a dry powder composition. In one embodiment, the process comprises adding more ethanol to the suspension to attain a density of the ethanol/water that is closer to the density of treprostinil; which causes the treprostinil to be less buoyant and more uniformly dispersed in the suspension. In another embodiment, the process comprises, adding treprostinil in a dry powder form to the suspension comprising the diketopiperazine particles with mixing until the powder is dissolved, and spray-drying or lyophilizing the suspension. In one embodiment, the process comprises the step of dissolving the treprostinil, derivative thereof or analog thereof in an ethanol solution prior to adding the solution to the diketopiperazine suspension.

In another embodiment, the process for manufacturing an inhalable dry powder composition for treating lung disease comprises; preparing a solution comprising a diketopiperazine in an aqueous ammonium solution up to about 30 wt %, or up to 40 wt %, filtering the solution formed; combining the solution with mixing with a filtered aqueous acetic acid solution in a high shear mixer to form a precipitate; concentrating the suspension by washing in multiple steps with deionized water; determining the percent solids in the suspension; freeze-drying or spray drying the suspension to form bulk FDKP microcrystalline powder. In an exemplary embodiment, dry powder comprising FDKP is resuspended in purified deionized water with or without ethanol; the pH of the suspension is adjusted to about 4.5, or to about 5.0. A solution of the active ingredient is made, for example, treprostinil, by dissolving the active ingredient in a solution comprising for example, from 70% to about 99.5% ethanol, or from 70% to about 99.5% absolute ethanol. The solution comprising the active ingredient is added to the particle suspension comprising the FDKP with mixing; followed by spray-drying the solution in a spray-drier.

The following examples illustrate some of the processes for making dry powders suitable for using with the inhalers described herein and data obtained from experiments using the dry powders.

### Example 1

Preparation of surfactant-free dry powder comprising FDKP microcrystalline powder for use with inhalers: In an example embodiment, surfactant free dry-powders comprising FDKP microcrystalline particles were prepared. Using a dual-feed high shear mixer, approximately equal masses of acetic acid solution (Table 1) and FDKP solution (Table 2) held at about 25° C.±5° C. were fed at 2000 psi through a 0.001-in² orifice to form a precipitate by homogenization. The precipitate was collected in deionized (DI) water of about equal temperature. The wt % content of FDKP microcrystallites in the suspension is about 2-3.5%. The concentration of FDKP in the suspension can be assayed for its solids content by an oven drying method. The FDKP microcrystallite suspension can be optionally washed by tangential flow filtration using deionized water. The FDKP microcrystallites can be optionally isolated by filtration, centrifugation, spray drying or lyophilization. The dry powder can be stored refrigerated or at room temperature, and/or used to add the active ingredients.

TABLE 1
Composition of Acetic Acid Solution

| Component | Component Range (wt. %) |
|---|---|
| Acetic Acid | 10.5-13.0 |
| Deionized Water | 87.0-89.5 |

TABLE 2
Composition of FDKP Solution

| Component | Component Range (wt. %) |
|---|---|
| FDKP | 2.5-6.25 |
| 30% NH4OH Solution | 1.6-1.75 |
| Deionized Water | 92-95.9 |

Dry powders (A, B, C and D) comprising microcrystalline particles made by the methods described above were tested for various characteristics, including surface area, water content and porosity measurements. Four different powders were used in this experiment. All powders tested had a residual water content of 0.4%. Table 2a demonstrates data obtained from the experiments.

TABLE 2a

| | | Pore | Pore |
|---|---|---|---|
| | | Volume BJH | Size BJH |
| | | Adsorption | Adsorption |
| | Surface Area | cumulative | average pore |
| | BET Surface | volume of | diameter |
| Powder ID | Area (m²/g) | pores (cm³/g) | (4 V/A) (nm) |
| A | 61.3 | 0.43 | 25.1 |
| B | 62.3 | 0.43 | 24.4 |
| C | 63.0 | 0.42 | 23.8 |
| D | 59.0 | 0.44 | 26.2 |

The data in Table 2a shows that the surface area of sprayed-dried, bulk dry powder comprising the microcrystalline particles of the samples tested ranged from 59 m²/g to 63 m²/g. The porosity data indicates that the microcrystalline particles are relatively porous, having average pore volumes of about 0.43 cm³/g and an average pore size ranging from about 23.8 nm to 26.2 nm as determined by BJH adsorption. The porosimetry data indicates that these particles differ from prior art FDKP microparticles which have been shown to have an average pore volume of about 0.36 cm³/g and average pore size from about 20 nm to about 22.6 nm.

### Example 2

Preparation of dry powder comprising microcrystalline FDKP particles containing treprostinil. A solution containing 0.2-1.0 wt % treprostinil in ethyl alcohol was added to a suspension of FDKP microcrystallites obtained as described in Example 1. The mixture was spray dried using a Buchi B290 spray-dryer equipped with a high efficiency cyclone. Nitrogen was used as the process gas (60 mm). Mixture was dried using 10-12% pump capacity, 90-100% aspiration rate, and an inlet temperature of 170-190° C. The weight % concentration of treprostinil in the resultant powder was 0.5-10%. Delivery efficiencies of these powders after discharge from a dry powder inhaler ranged between approximately 50% and 70%.

### Example 3

Use of treprostinil-fumaryl diketopiperazine (TIP) composition in healthy subjects.

This study was an open-label, single ascending dose study in 36 healthy normal volunteers that were sequentially assigned to 6 cohorts receiving single doses of TIP (30, 60, 90, 120, 150, and 180 μg). The safety and tolerability of the dry powder compositions comprising treprostinil was evaluated in each sequential cohort prior to escalating the dose for the next cohort using a dry powder inhaler system comprising a cartridge dose in a single inhalation. Blood samples were obtained before administration of the composition and at selected times through 480 minutes post-dose. Blood samples were analyzed for treprostinil using a validated analytical method and PK parameters were calculated using non-compartmental methods.

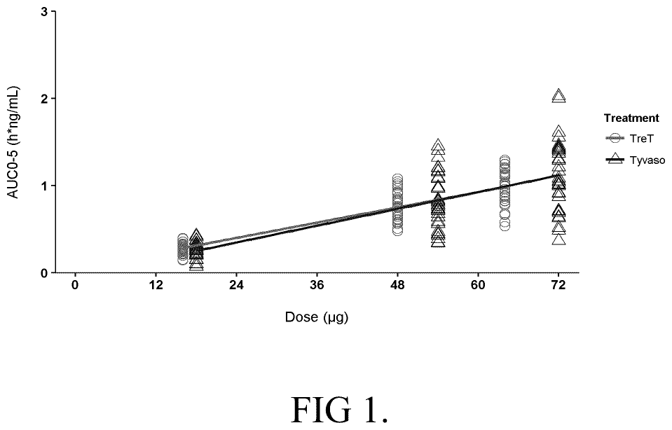

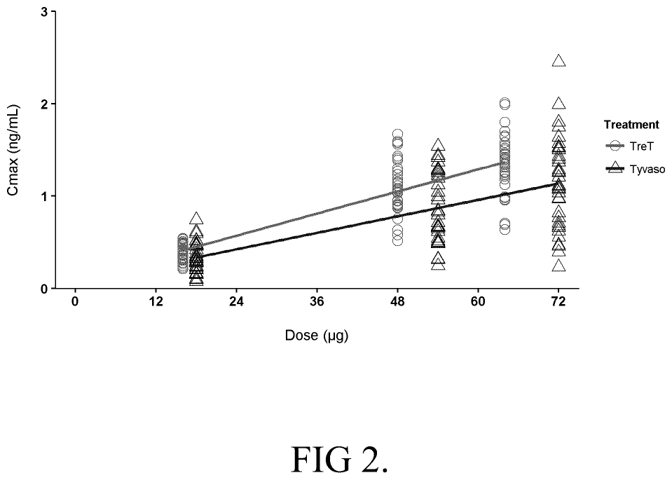

A total of 36 individuals were randomized and dosed. There were no severe adverse events, serious adverse events, or deaths during this study. No adverse events led to a subject's early termination. The most frequently reported adverse events were cough (n=11, 30.6%) and headache (n=8, 22%). Bioanalysis data confirmed that the treprostinil plasma concentrations and exposure for treprostinil, achieved clinically relevant concentrations comparable to those observed in historical TYVASO® single dose clinical studies (Table 3, FIGS. 1 and 2). Table 3 shows study design for dosing comparing TYVASO® versus a single inhalation of a cartridge of TIP using a DPI. FIG. 1 demonstrates the AUC0-5 and dose for subjects treated with TIP and TYVASO®. FIG. 2 shows the Cmax (ng/mL) for treprostinil for the various amounts of dose administered as described in Table 3 for TIP and TYVASO®. The data in FIGS. 1 and 2 indicate Cₘₐₓ and AUC for treprostinil, increased in a linear manner with increasing dose. The results are also shown in Table 4.

TABLE 3

| TYVASO ® Dose | TreT Dose |
|---|---|
| 18 μg (3 nebulizer breaths) | 16 μg cartridge |
| 54 μg (9 nebulizer breaths) | 48 μg cartridge |
| 72 μg (12 nebulizer breaths) | 64 μg cartridge |

TABLE 4

| | | | | Geometric LSM | |
|---|---|---|---|---|---|
| | | Geometric LSM | Geometric LSM | Ratio (%) | 90% Confidence |
| Comparison | Parameter | (TreT ) [CV %] | (Tyvaso) [CV %] | [TreT/Tyvaso] | Interval |
| TreT 16 μg vs. | AUC0-5 | 0.268 [24.1%] | 0.233 [44.1%] | 115 | (104.59, 127.42) |
| TYVASO ® 18 μg | Cmax | 0.377 [26.6%] | 0.291 [59.8%] | 130 | (115.55, 145.95) |
| TreT 48 μg vs. | AUC0-5 | 0.766 [21.8%] | 0.757 [42.5%] | 101 | (91.63, 111.65) |
| TYVASO ® 54 μg | Cmax | 1.07 [28.9%] | 0.764 [53.4%] | 139 | (124.13, 156.73) |
| TreT 64 μg vs. | AUC0-5 | 0.937 [23.8%] | 1.02 [41.9%] | 91.5 | (83.16, 100.78) |
| TYVASO ® 72 μg | Cmax | 1.27 [28.5%] | 1.02 [54.7%] | 124 | (110.56, 139.61) |

The PK study data illustrates that AUC0-5 was generally comparable for each TIP-DPI and TYVASO® dose level. The data also shows Cmax values for TIP-DPI were slightly higher than TYVASO® Cmax values across dose comparisons. The data above illustrates that TIP DPI is more efficient in delivering Treprostinil to individuals in a single inhalation than in nebulized form of TYVASO®, which requires multiple inhalations per session, and the DPI form requires less amounts of Treprostinil per dose.

Adverse events profile is consistent with known prostacyclin effects and previous studies of TYVASO®. Between-subject variability for both AUC0-5 and Cmax was approximately two-fold less for TIP-DPI compared to TYVASO®. AUC0-5 and Cmax for TIP-DPI and TYVASO® increased in an approximately dose-proportional manner and the median Tmax was approximately 10 minutes for TIP-DPI and 10 to 15 minutes with TYVASO®.

### Example 4

Use of treprostinil-fumaryl diketopiperazine inhalation powder (TIP) in subjects in with pulmonary arterial hypertension (PAH) which were treated with treprostinil nebulization (TYVASO®). This study design was a comparative study which evaluated the safety and tolerability of TIP in PAH patients. The study also evaluated systemic exposure and pharmacokinetics (PK) of treprostinil in 51 subjects with PAH (WHO Functional Class I (11.8%), II (60.8%) and III (27.5%), ranging in age from 23-82 years. There were 43 females and 8 male subjects in the study. Treprostinil was delivered by nebulization (TYVASO®), or a dry powder inhaler (TIP). TIP was administered to the subject with a DPI (DREAMBOAT® inhaler, MannKind Corp.) in a single inhalation per cartridge dose. The subjects (51) were administered 32 μg, 48 μg, and 64 μg of treprostinil twice daily dose for a period of three weeks and 49 subjects continued treatment. Serial pharmacokinetic sampling were obtained at baseline and at 3 weeks after start of the study, and treatment continued twice daily for the remaining term of the study. Follow-up clinic visits occurred every 8 weeks post study start (see Table 5 below).

TABLE 5
Treprostinil Dosing

| TYVASO ® Dose (QID) | TIP Dose (QID) | DPI Device Content |
|---|---|---|
| 6 to 7 breaths | 32 μg | 32 μg cartridge |
| 8 to 10 breaths | 48 μg | 48 μg cartridge |
| 11 to 12 breaths | 64 μg | 32 μg + 32 μg cartridges |

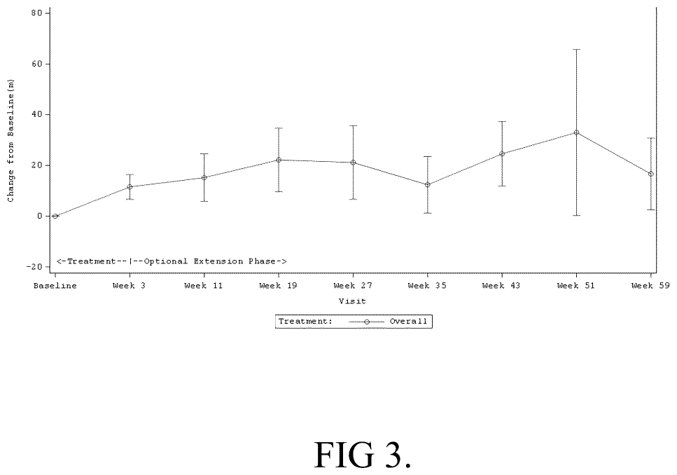

Baseline subject physical characteristics were measured prior to the treatment period as assessed by the 6-minute walk distance test (6MWD) for patient using nebulized treprostinil, which was also measured at various intervals and at the end of the study. FIG. 3 shows the results of the study treatment. As can be seen, the change in baseline in 6MWD test for TIP overall demonstrates a significant improvement (8.0 m increase; p=0.0217) at week 3 of treatment. The improvements in 6MWD for TIP overall were sustained in the subjects which were treated for 59 weeks. Patients (95.7%) reported overall satisfaction with TIP-DPI when compared to the treprostinil nebulizer.

At week 3 and week 11, patients were given a PAH-SYMPACT, well validated patient-reported outcome questionnaire to assess PAH symptoms and effects. The PAH-SYMPACT contains four domains (Cardiopulmonary symptoms, cardiovascular symptoms, physical impacts, cognitive/emotional impacts) and was given at baseline, week 3 and week 11 of the study. The data revealed a trend of improvement at both week 3 and week 11 for subjects receiving TIP-DPI. Mean change from Baseline was lower for all domain scores of the PAH-SYMPACT at both weeks (range: −0.05 to −0.22), with significant improvements for physical impacts scores (range: −1.1 to 1.0; p=0.0438) and cognitive/emotional impacts (range: −1.3 to 0.5; p=0.0048) at Week 3. The study report also showed a decrease in adverse events during the treatment phase for those patients treated with TIP versus standard therapy with TYVASO® for the entire study (see Tables 6 and 7).

TABLE 6

| | Treatment Phase Dose | | | | | | | |
|---|---|---|---|---|---|---|---|---|
| | 32 mcg | 48 mcg | 64 mcg | Overall | TRIUMPH | | | |
| Preferred | N = 2 | N = 27 | N = 22 | N = 51 | Tyvaso | | Placebo | |
| Term | n (%) | n (%) | n (%) | n (%) | n (%) | | n (%) | |
| Cough | 0 | 9 (33.3) | 4 (18.2) | 13 (25.5) | 62 | (54) | 35 | (29) |
| Headache | 0 | 4 (14.8) | 4 (18.2) | 8 (15.7) | 47 | (41) | 27 | (23) |
| Dyspnoea | 0 | 2 (7.4) | 1 (4.5) | 3 (5.9) | 6 | (5) | 6 | (5) |
| Flushing | 0 | 1 (3.7) | 1 (4.5) | 2 (3.9) | 17 | (15) | 1 | (<1) |
| Nausea | 0 | 2 (7.4) | 0 | 2 (3.9) | 22 | (19) | 13 | (11) |
| Throat | 0 | 1 (3.7) | 1 (4.5) | 2 (3.9) | 29 | (25)* | 17 | (14)* |
| irritation | | | | | | | | |

TABLE 7

| | TreT/TIP Dose in Treatment Phase | | | |
|---|---|---|---|---|
| | 32 mcg | 48 mcg | 64 mcg | Overall |
| | N = 2 | N = 26 | N = 21 | N = 49 |
| Preferred Term | n (%) | n (%) | n (%) | n (%) |
| Cough | 0 | 3 (11.5) | 2 (9.5) | 5 (10.2) |
| Dyspnoea | 1 (50.0) | 2 (7.7) | 2 (9.5) | 5 (10.2) |
| Headache | 0 | 2 (7.7) | 2 (9.5) | 4 (8.2) |
| Diarrhoea | 0 | 1 (3.8) | 2 (9.5) | 3 (6.1) |
| Pneumonia | 0 | 2 (7.7) | 1 (4.8) | 3 (6.1) |
| Arthralgia | 0 | 2 (7.7) | 1 (4.8) | 3 (6.1) |
| Dizziness | 0 | 2 (7.7) | 1 (4.8) | 3 (6.1) |

Overall, TreT/treprostinil was safe and well-tolerated and produced clinically relevant concentrations of treprostinil when inhaled as a dry powder.

The preceding disclosures are illustrative embodiments. It should be appreciated by those of skill in the art that the devices, techniques and methods disclosed herein elucidate representative embodiments that function well in the practice of the present disclosure. However, those of skill in the art should, in light of the present disclosure, appreciate that many changes can be made in the specific embodiments that are disclosed and still obtain a like or similar result without departing from the spirit and scope of the invention.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope are approximations, the numerical values set forth in the specific examples are reported as precisely as possible. Any numerical value, however, inherently contains certain errors necessarily resulting from the standard deviation found in their respective testing measurements.

The terms “a” and “an” and “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope otherwise claimed. No language in the specification should be construed as indicating any non-claimed element essential to the practice of the invention.

The use of the term “or” in the claims is used to mean “and/or” unless explicitly indicated to refer to alternatives only or the alternatives are mutually exclusive, although the disclosure supports a definition that refers to only alternatives and “and/or.”

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments are described herein, including the best mode known to the inventors for carrying out the invention. Of course, variations on those preferred embodiments will become apparent to those of ordinary skill in the art upon reading the foregoing description. The inventor expects those of ordinary skill in the art to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments so claimed are inherently or expressly described and enabled herein.

Furthermore, numerous references have been made to patents and printed publications throughout this specification. Each of the above cited references and printed publications are herein individually incorporated by reference in their entirety.

Further, it is to be understood that the embodiments disclosed herein are illustrative of the principles of the present invention. Other modifications that may be employed are within the scope of the invention. Thus, by way of example, but not of limitation, alternative configurations may be utilized in accordance with the teachings herein. Accordingly, the present invention is not limited to that precisely as shown and described.

We claim:

1. A process for making an inhalable dry powder composition comprising,
preparing a suspension of microcrystalline particles of (E)-3,6-bis[4-(N-carbonyl 2-propenyl)amidobutyl]-2,5-diketopiperazine in an aqueous ammonia solution and combining an acetic acid solution in a high shear mixer at a temperature ranging from 13° C. to about 20° C. while mixing in a high shear mixer to form a suspension;
washing the suspension in water; pelletizing the suspension in a cryogranulator, and drying the suspension in a lyophilizer to collect the microcrystalline particles of the diketopiperazine;
resuspending the microcrystalline particles of the diketopiperazine in in a solution of deionized water and ethanol; preparing a hydrophobic compound in in a solution of about 70% to about 100% ethanol, and adding the solution to the suspension with mixing and drying the suspension.

2. The process of claim 1, wherein the hydrophobic compound is a prostaglandin.

3. The process of claim 2, wherein the prostaglandin is treprostinil, a derivative thereof or an analog thereof.

4. The process of claim 1, wherein the resuspending step comprises dried crystalline particles of a diketopiperazine in a solution of deionized water and an alcohol that contains from about 0.2% to about 2% solid in the suspension, or from about 1% to about 4% solid in the suspension, or from about 1% to about 10% solid in the suspension.

5. An inhalable dry powder composition made by the process of claim 1.

6. The inhalable dry powder composition of claim 5, wherein the hydrophobic compound is a prostaglandin.

7. The inhalable dry powder composition of claim 6, wherein the prostaglandin is treprostinil, a derivative thereof or an analog thereof.

8. A cartridge comprising an inhalable dry powder composition made by the process of claim 1.

9. The cartridge of claim 8, wherein the hydrophobic compound is a prostaglandin.

10. The cartridge of claim 9, wherein the prostaglandin is treprostinil, a derivative thereof or an analog thereof.

11. An inhaler comprising a hydrophobic compound made by the process comprising,
preparing a suspension of microcrystalline particles of (E)-3,6-bis[4-(N-carbonyl 2-propenyl)amidobutyl]-2,5-diketopiperazine in an aqueous ammonia solution and combining an acetic acid solution in a high shear mixer at a temperature ranging from 13° C. to about 20° C. while mixing in a high shear mixer to form a suspension;
washing the suspension in water; pelletizing the suspension in a cryogranulator, and drying the suspension in a lyophilizer to collect the microcrystalline particles of the diketopiperazine;
resuspending the microcrystalline particles of the diketopiperazine in in a solution of deionized water and ethanol; preparing the hydrophobic compound in in a solution of about 70% to about 100% ethanol, and adding the solution to the suspension with mixing and drying the suspension.

12. The inhaler of claim 11, wherein the hydrophobic compound is a prostaglandin.

13. The inhaler of claim 12, wherein the prostaglandin is treprostinil, a derivative thereof or an analog thereof.

14. The inhaler of claim 11, wherein the resuspending step comprises dried crystalline particles of a diketopiperazine in a solution of deionized water and an alcohol that contains from about 0.2% to about 2% solid in the suspension, or from about 1% to about 4% solid in the suspension, or from about 1% to about 10% solid in the suspension.

15. A method of treating pulmonary hypertension comprising administering to a patient in need of treatment an inhalable, substantially crystalline dry powder composition comprising a treprostinil dose in an amount of up to 200 μg and one or more pharmaceutically acceptable salts thereof and a pharmaceutically acceptable carrier and/or excipient, wherein said administering comprises at least one to four inhalations per day.

16. The method of claim 15, wherein the substantially crystalline dry powder composition comprises a diketopiperazine.

17. The method of claim 15, wherein the diketopiperazine is (E)-3,6-bis[4-(N-carbonyl-2-propenyl)amidobutyl]-2,5-diketopiperazine.
